(19) United States
(12) Patent Application Publication
(10) Pub. No.: US 2015/0079676 A1
(43) Pub. Date: Mar. 19, 2015

(54) METHOD AND APPARATUS FOR THE ISOLATION OF MOTILE SPERM

(71) Applicant: AUCKLAND UNISERVICES LIMITED, Auckland (NZ)

(72) Inventors: Bryon Elmer Wright, Auckland (NZ); Mark Philip Green, Melbourne (AU)

(21) Appl. No.: 14/382,046

(22) PCT Filed: Feb. 28, 2013
(86) PCT No.: PCT/NZ2013/000026

§ 371 Date: Aug. 29, 2014

(30) Foreign Application Priority Data
Feb. 29, 2012 (NZ) 598506

(51) Int. Cl. C12N 5/076; B01L 3/00

(52) U.S. Cl. CPC C12N 5/061; B01L 3/502746; B01L 3/502753
USPC 435/366; 435/325; 422/527

(57) ABSTRACT

A system for separating motile and non-motile sperm comprises a microvolume into which a fluid containing sperm is delivered, at least partly defined by a wall which includes a termination or a change in angle away from the microvolume, defining an exit from the microvolume. Sperm delivered into the microvolume in a fluid that are motile move entrained along the wall and leave the wall at the termination with an outward turn to or towards a collection reservoir or passage from the microvolume. Typically the system is implemented on a microfluidic chip.

### FIELD OF INVENTION

The invention pertains to a method for the sorting and collection of motile sperm from a liquid containing both motile sperm and non-motile particles into another liquid, and to apparatus suitable therefor.

### BACKGROUND

Assisted reproductive technologies (ART) are used to artificially enhance or augment the reproduction process to benefit human medicine or the agricultural industry. A fundamental process in ART is the collection and processing of a sperm collection to obtain an optimised sperm product. The optimised sperm product can then be used immediately or stored. This is done by using one or more of a number of sperm processing techniques; these include a wash in medium, filtration, swim-up, as well as density gradient centrifugation (commonly referred to as Percoll-type). Aside from a simple wash, all require a laboratory environment and equipment, with careful control of temperature, osmolality, and other processing conditions.

The two main reproductive technologies that rely on the processing of sperm are artificial insemination (AI) and in vitro fertilisation (IVF). AI is the most commonly undertaken ART frequently practiced in numerous species, including humans, that has documented advantages over natural reproduction. In livestock species such as cattle, AI is a large, well established industry that uses ART to manage and improve herd genetics for commercial benefits. The second especially significant and growing ART is the sorting of sperm cells for use in human IVF, including the specialised practice of intracytoplasmic sperm injection (ICSI) that is undertaken when the availability of viable sperm is very low, i.e. below 1 million/ml; the standard threshold used in IVF, as the fertilisation rate is known to decrease considerably below this concentration.

Sperm processing intends to increase the effectiveness of the ART procedures that follow. The main criterion is the isolation of sperm capable of achieving optimal fertilisation and pregnancy rates. For IVF, the most desirable sperm exhibit motility as sperm motility correlates highly with successful fertilisation and therefore IVF success. The World Health Organisation (WHO) has set guidelines for the evaluation of sperm motility and a minimum of 40% motile sperm is recommended for an IVF procedure and less requires the more technically-challenging and costly ICSI procedure. Even for healthy samples with high motility, sperm are enriched according to motility in order to give the greatest chance of fertilisation.

Therefore, the primary results from common sperm processing techniques are the enrichment of motile sperm concentrations and improvement in the motility grade of processed sperm. Other beneficial results of common sperm processing techniques might include changes to the medium favourable to ART success and an increase in sperm concentration. Medium exchange is particularly important for fresh semen samples, initially to remove it from the seminal fluid, and secondly to avoid oxidative stress from immotile components of semen and resultant problems with sperm capacitation and hyperactivation that have been shown to lead to decreased IVF fertilisation rates.

Current sperm processing techniques used to prepare sperm for ART involve a number of manual handling steps and centrifugation, thus are often labourious. These manipulations are known to be detrimental to sperm quality due to exposure to environmental stressors such as temperature and osmolality changes, as well as the oxidative species generated by suboptimal sperm during the preparation process, not to mention the forces involved in centrifugation that result in physical degradation and DNA fragmentation. Recently, there has been increased reporting on how poor sperm quality, especially as a result of DNA damage, decreases IVF success rates. Additionally, the current use of manual handling increases the likelihood of operator errors in processing.

### SUMMARY OF THE INVENTION

In broad terms in one aspect the invention comprises a method of separating motile and nonmotile sperm which comprises
delivering a fluid containing sperm into a microvolume at least partially defined by a wall which includes a wall termination or a change in angle away from the microvolume (herein: wall termination), and
allowing at least some motile sperm to move along said wall and to exit the microvolume by changing direction away from the microvolume at or near the wall termination, to or towards collection of motile sperm.

In broad terms in another aspect the invention comprises a system for separating motile and non-motile sperm, which comprises a microvolume into which a fluid containing sperm can be delivered, at least partly defined by a wall which includes a termination or a change in angle away from the microvolume (‘wall termination’), the wall termination at least in part defining an exit from the microvolume to or towards a collection reservoir or passage from the microvolume for motile sperm.

Preferably the microvolume is further defined by at least one further wall termination adjacent the first said wall termination, the two or more opposing wall terminations defining between them an opening (herein sometimes: trap) from the microvolume. Preferably the opening from the microvolume is to a collection reservoir for motile sperm, optionally via a passage between the opening and the collection reservoir.

Preferably the collection reservoir is defined in least in part by a concavely arcuate wall portion to assist in retaining motile sperm within the collection reservoir. Preferably the concavely arcuate wall comprises more than one half of a boundary in at least one dimension, defining the collection reservoir.

Preferably the microvolume has a planar form i.e. a length and/or width greater than a depth. The length of the microvolume is generally much greater than either the width or the depth, to form an extended channel structure. In a preferred form the microvolume has a width in the range 10 to 5000 microns, or 10 to 500 microns, or 50 to 500 microns, and a depth in the range 5 to 250 or 1000 microns. Preferably the width of the microvolume is greater than the depth of the microvolume, preferably at least two or at least five or at least ten times greater than the width or depth of the microvolume. The wall(s) comprising the wall termination(s) is/are in the depth of the planar microvolume, and preferably extend(s) in a length of the volume.

Preferably a change in angle at said wall termination(s) is between about 1 or 20° and approaching 180°, or between about 60° and approaching 180°, or between about 90° and 150°. Preferably the change in angle at said wall termination(s) defines an opening or trap dimension in the length of the microchannel of at least 10 microns, up to 250, 500 or 750 microns for example.

Preferably the maximum distance that a motile sperm introduced into the microvolume must travel along the shortest possible path before encountering a wall, becoming entrained, and encountering a wall termination along the same wall should be between 1 and 5000 microns, or between 50 and 2000 microns.

Preferably the system includes a control system arranged to control delivery of fluid containing sperm in a fluid into the microvolume, subsequent maintenance of a period of substantially no-flow conditions in the microvolumel, and recovery or delivery of fluid containing the motile sperm from the microvolume after a period of said substantially no-flow conditions.

In broad terms in another aspect the invention comprises a method of separating motile and non-motile sperm which comprises
delivering a fluid containing the sperm in a fluid into a microchannel comprising a wall which includes a wall termination or a change in angle away from the microchannel (wall termination),
allowing motile sperm to move in substantially no-flow conditions in the microchannel along the wall and to exit the microchannel by changing direction away from the microchannel at or near the wall termination, to or towards a collection reservoir, and
recovery of fluid containing the sorted sperm from the collection reservoir.

Typically the step of allowing motile sperm to move in substantially no flow conditions includes maintaining the substantially no flow conditions for at least 10 or 20 seconds, more preferably at least 60 seconds (to separate of the order of 75% of motile sperm for example), or for more than 60 seconds, up to for example 90 seconds.

The method may also comprise flushing the microchannel of remaining fluid containing any nonmotile sperm, before or after the step of recovering fluid containing the motile sperm from the reservoir. The step of recovering the motile sperm may include recovering the motile sperm from the reservoir back into and through the microchannel. Alternatively fluid containing motile sperm in the reservoir may be recovered via another channel from the reservoir.

Embodiments of the method include priming the microfluidic structures with a collection medium, injecting the initial sperm-containing medium, and a sorting phase in which static fluid flow conditions are established and progressively motile sperm are entrained at microfluidic interfaces (wall(s)). These entrained motile sperm are partitioned through trap structures, away from the main microfluidic channel, and into collection reservoirs containing the collection medium. In the sorting phase cessation of the flow allows motile sperm to actively swim to the wall(s) of a channel and track using their innate aptitude along surfaces to self-sort through traps into side reservoirs in to fresh medium. Lesser-motile particles within the channel are then actively flushed out and replaced with new fluid. Optionally the method may include concentrating through repeated cycles of this process, before motile sperm are actively flushed from the reservoir and collected. The specific angles and design of the traps is chosen to reduce exit of sperm back into the main channel. Coordinated valving is then used to remove the initial medium including immotile components, and collect the progressively motile sperm from the collection reservoirs in the collection medium.

A system of the invention for sorting sperm may have at least one set of functional structures including the trap, reservoir, and injection or microchannel, or arrays of multiple numbers of these structures to increase throughput. The system is preferably embodied in a small microfluidic device or chip prepared by micromachining or polymer processing techniques to form the microfluidic structures, and comprises supporting pumps, valving and instrumentation.

In broad terms in a further aspect the invention comprises a multichannel microfluidic array for separating or concentrating motile sperm, said array comprising:
an inlet port for sperm;
an inlet port for carrier medium;
an exit port for carrier medium and unseparated or non-motile sperm;
a delivery port for flushing medium;
a recovery port for separated or concentrated motile sperm;
a plurality of parallel microchannels, each microchannel including side walls, and each side wall having a plurality of wall terminations; each wall termination forming an entrance to a motile sperm collection reservoir;
a plurality of sperm collection reservoirs interconnectable between the delivery port and the recovery port through the microchannels;
valve means for controlling fluid flow either from the inlet ports to the exit port or from the delivery port to the recovery port
manifold systems to distribute and recover fluids substantially evenly to and from the microchannels and/or collection reservoirs.

### BRIEF DESCRIPTION OF THE DRAWINGS

The invention is further described with reference to the accompanying figures in which:

FIG. 1a schematically illustrates the edge guidance principle used to separate motile from nonmotile sperm and FIG. 1b schematically illustrates a first embodiment of the invention,

FIGS. 2a-2c schematically illustrate a second embodiment of the invention comprising a double wall termination trap, and a reservoir,

FIGS. 3a-3e schematically illustrate steps of a method of the invention,

FIGS. 4a and 4b are schematic perspective cutaway views from above of the microchannel and trap of FIG. 3, and of an alternative embodiment of a microchannel and trap, respectively,

FIG. 5 schematically illustrates a further embodiment of the invention comprising a double wall termination trap and two associated reservoirs,

FIG. 6a is a schematic perspective cutaway view of another double wall termination trap embodiment with two reservoirs and two collection outlets, and FIG. 6b is a schematic cutaway perspective view of another double wall termination trap with two reservoirs and a single collection outlet,

FIG. 7a schematically illustrates a system of the invention for two traps on a microfluidic chip, and FIG. 7b schematically illustrates a system of the invention for more than two traps on a microfluidic chip, and

FIG. 8 schematically illustrates a 1024-trap system of the invention on a microfluidic chip.

### DETAILED DESCRIPTION OF THE PREFERRED EMBODIMENTS

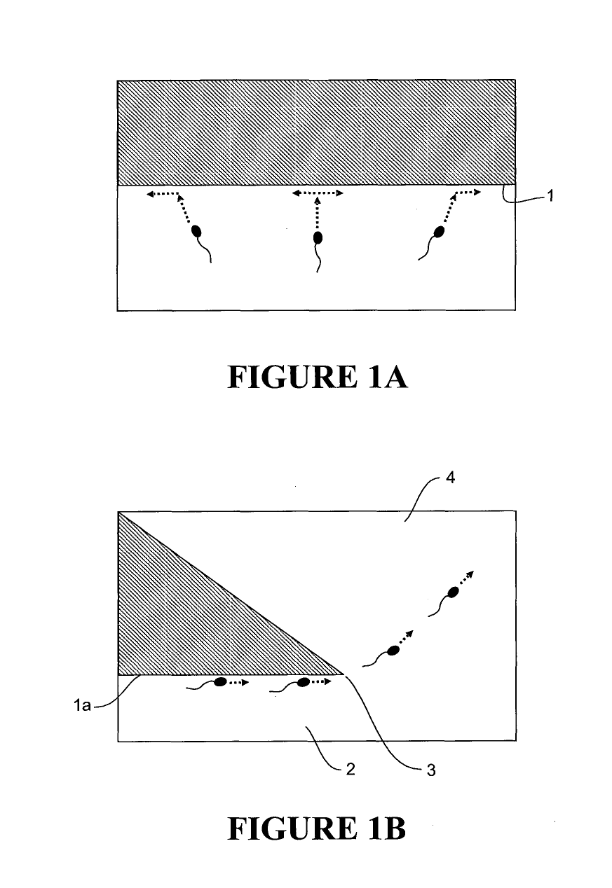

Referring to FIG. 1a, motile sperm (black headed) with progressive motion that encounter a wall 1 will tend to turn and move along the wall (become ‘edge-entrained’) in a direction depending on the initial approach angle, as shown. The wall then acts as a guide for the sperm motion in that edge-entrained sperm will tend to move along the wall, unless perturbed.

FIG. 1b schematically illustrates a system and method for separating motile and non-motile sperm of a first and simplest embodiment of the invention. A microvolume 2 typically provided on a micro fluidic chip, such as a microchannel, is defined at least in part by wall or wall part 1a. The wall or wall part 1a ends at termination 3. Sperm delivered into the microvolume 2 (in a fluid) that are motile (black headed) and encountering wall 1a will move along the wall and sperm so entrained will leave the wall at termination 3 in a predictable way—with an outward turn away from the microlinear channel 2, typically of approximately 135°. Those motile sperm can be collected at 4. At least some non-motile sperm may remain in the microvolume.

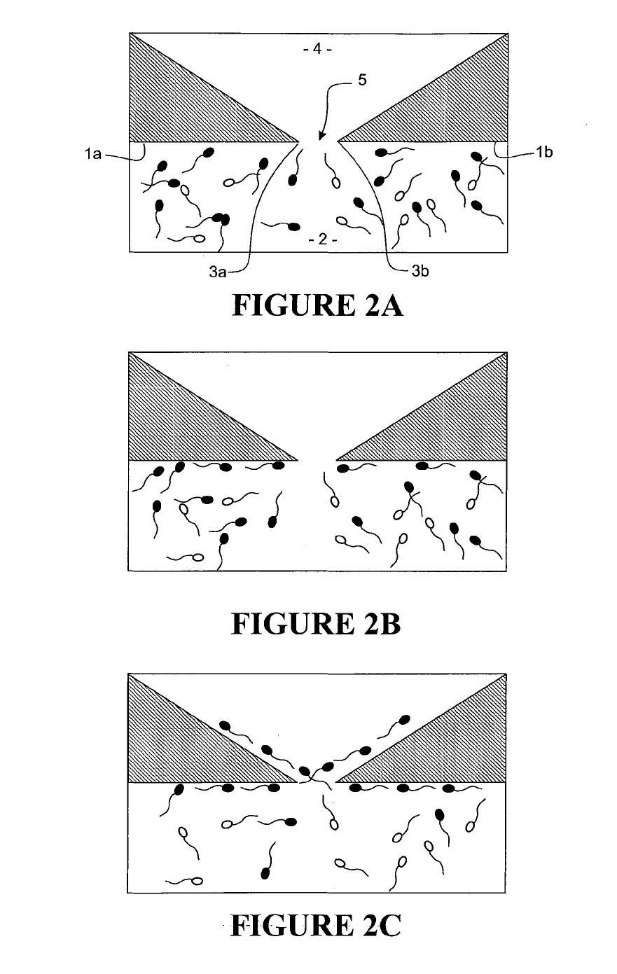

FIG. 2 schematically illustrates a second embodiment, in which microchannel 2 includes adjacent wall portions 1a and 1b with opposing wall terminations 3a and 3b, which define between them an opening 5 or trap (reference numbers provided on FIG. 2a only, for clarity). Following injection into the microchannel 2 of an initially disorganised mix of motile sperm (black heads) and non-motile sperm (white heads) and other components of a complex liquid such as semen, and stopping of the bulk liquid flow in the microchannel, no special organisation of sperm is necessarily present as shown in FIG. 2a. The zero-flow condition is maintained for a period, during which sperm will move along their initial trajectories with some variation in motion such that all will eventually encounter the walls 1a and 1b, with many becoming entrained along the walls, as shown in FIG. 2b. Those entrained motile sperm will leave the walls 1a and 1b at terminations 3a and 3b, and pass through the trap 5 to a collection reservoir genetically indicated at 4—see FIG. 2c. All or much non-motile sperm and other components of the liquid remain in the microchannel 2 or distribute relatively slowly as per diffusive transport.

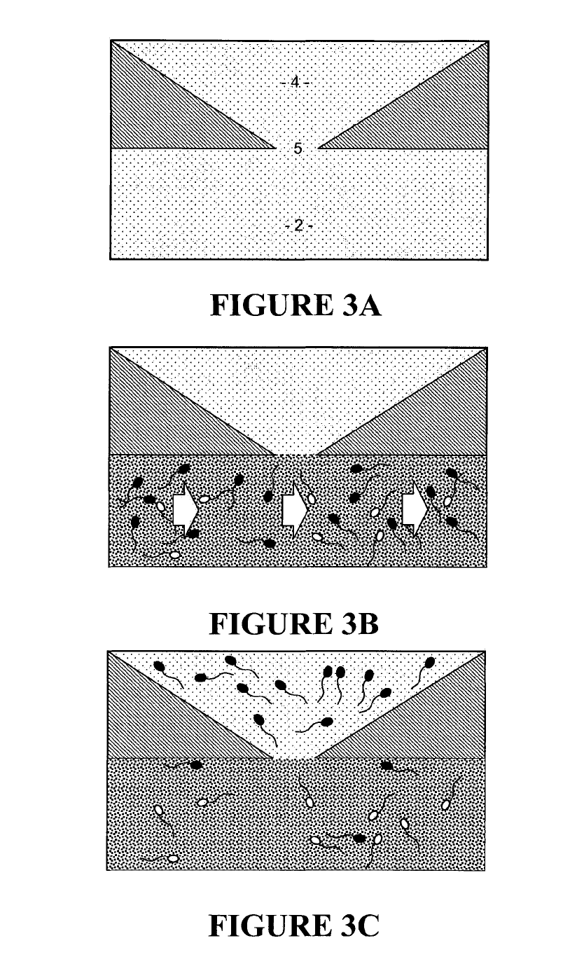

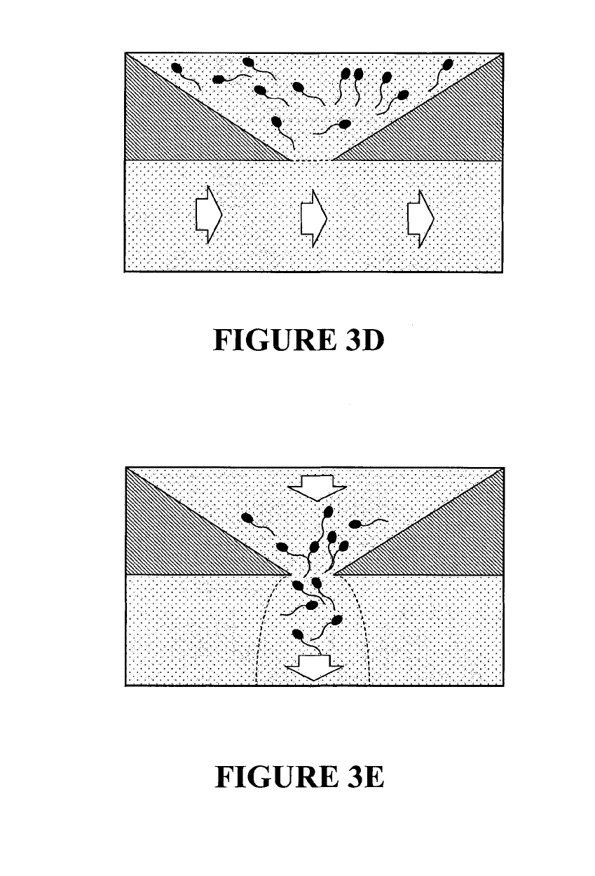

The steps of a method of an embodiment of the invention are schematically illustrated in FIGS. 3a-3e, in which in general the same reference numbers as in FIG. 2 indicate the same elements. The system also comprises valves to allow cycles of initial purge, sperm injection, sorting, purging, and collection. Initially all structures including the microchannel 2 and reservoir(s) 4 are filled with a solution suitable to the maintenance of sperm in vitro (“medium”), as shown in FIG. 3a. As shown in FIG. 3b, the sperm—containing solution is then injected into the microchannel 2 as indicated by the arrows, with the outlet of reservoir 4 close in order to create a laminar interface at trap 5 such that the initial medium is retained in the reservoir 4. All flow is 5 then stopped for a period sufficient to allow at least a major fraction of the injected sperm to sort as described above—see FIG. 3c, until the reservoir 4 contains predominantly progressively motile sperm. The remaining sperm-containing solution in the injection channel is then replaced with medium as shown in FIG. 3d, in which the arrows again indicate in flow of fresh medium. The motile sperm are then collected by opening the collection reservoir 4 to a purging flow of medium as indicated by the arrows, leaving the system as shown in FIG. 3a and ready for another injection cycle.

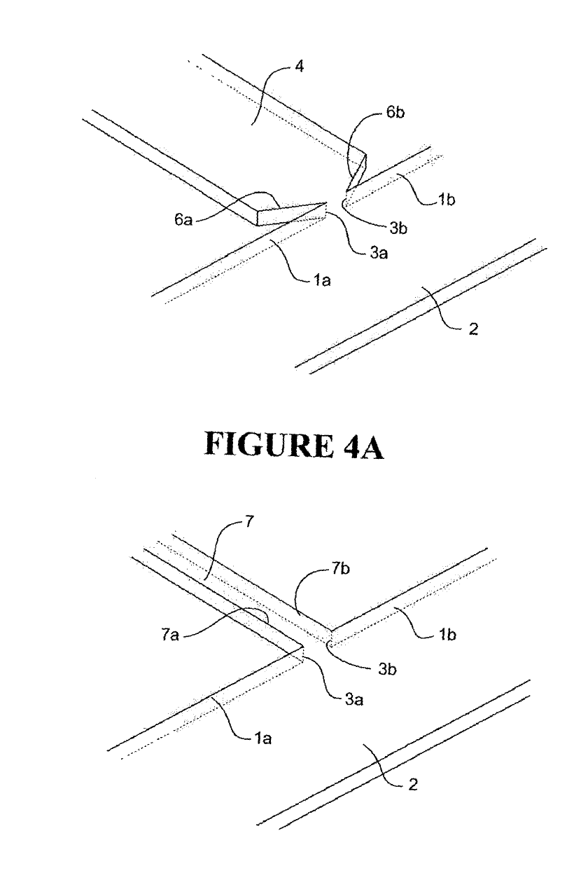

FIG. 4a is a schematic perspective cutaway view from above the microchannel 2 and trap 5 leading to reservoir 4, of FIGS. 2 and 3. As shown, microchannel walls 1a and 1b terminate at opposing terminations 3a and 3b defining trap 5 between them, and at which the walls 1a and 1b meet reverse angled walls 6a and 6b opening to side walls of reservoir 4. In this and other embodiments this reverse angle may be between greater than 0 or about 60° to approaching 180°, or between about 110 or 120° and 150 or 160°. In the embodiment shown it is about 150°. The angle may be radiused, with a radius in the range 1 to 100 microns or about 20 microns for example. The trap 5 may have a dimension in the length of the microchannel of typically 5 or 10 to 200 or 250 microns, such as about 75 microns for example.

FIG. 4b is a view similar to that of FIG. 4a but of an alternative embodiment, in which trap 5 from the microchannel leads to a channel 7 to a reservoir or other collection for the fluid containing motile sperm. The walls 1a and 1b at terminations 3a and 3b meet walls 7a and 7b of the channel 7 at substantially at 90° in this embodiment as shown. The angle may be radiused, with a radius in the range 1 to 50 or 100 microns or about 5 or 20 microns for example.

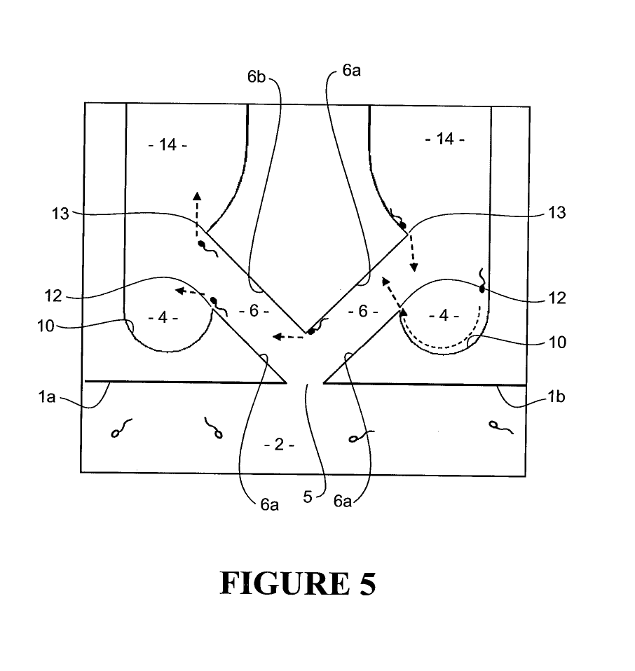

FIG. 5 is a schematic cross-section plan view of a microfluidic chip of another embodiment of the invention. Again, in general, the same reference numerals as in prior figures indicate the same elements. Microchannel 2 comprises a trap opening 5 (between opposing wall terminations) through which motile sperm entrained along microchannel side walls 1a and 1b will pass. The microfluidic system also comprises complementary channels 6 to collection reservoirs 4. Each reservoir 4 also comprises a sperm collection outlet or outlet channel 14. Each collection reservoir 4 is defined substantially by a concavely arcuate wall 10. At the opening to each reservoir 4 from the channel 6 is provided a further wall termination 12. Sperm moving along wall 6a of channel 6 towards reservoir 4 will tend to change angle into the reservoir 4 at termination 12 as shown on the left hand side of FIG. 5. Sperm moving in the channel 6 entrained against opposite channel wall 6b, on encountering an edge termination 13 will tend to change angle to move towards sperm collection outlet 14, as shown on the right hand side of FIG. 5. Sperm moving in the collection reservoir 4 entrained against the arcuate wall will tend to circulate within the reservoir 4 as indicated on the right hand side of FIG. 4, until collected. Any sperm moving from the collection reservoir encountering edge 12 from within the reservoir will tend to meet the opposite channel wall and then move towards outlet channel 4, as also indicated on the right hand side of FIG. 5. Thus the likelihood of sperm returning to the microchannel 2 is minimised. In use the microfluidic sperm sorting system may be operated according to the method described above in relation to FIG. 3, for example.

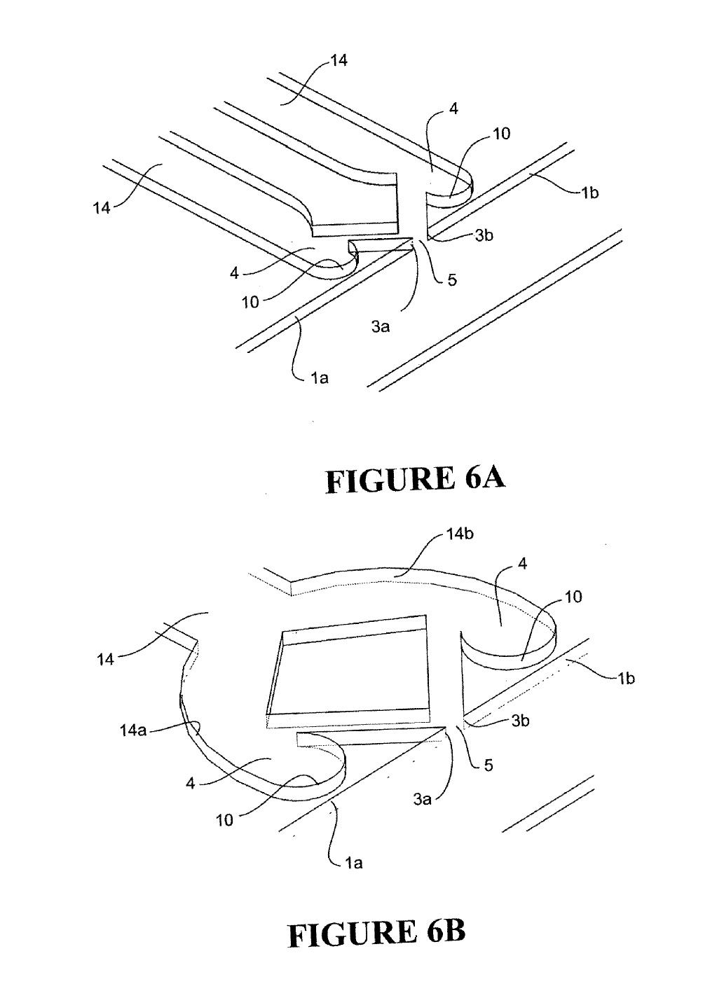

FIG. 6a is a schematic cutaway perspective view from above of the embodiment shown in FIG. 5. In this embodiment a single trap opening 5 leads to two outlet reservoirs 14.

FIG. 6b is a schematic cutaway perspective view from above of an alternative embodiment of a separation and reservoir system similar to that of FIG. 5 but comprising a common outlet channel 14. Curved walls 14a and 14b extend from reservoirs 4 on either side to channel 14 as shown.

In addition, more than two outlet reservoirs 14 can be connected to a single collection reservoir 4, which can in turn have one or more trap openings 5.

In the embodiments described and illustrated in the figures, the microstructure has a planar form with in-plane length and width greater than depth transverse to the plane. In alternative embodiments, the depth may be greater than the length and/or width of the microchannel and reservoir and other cavities of the microsystem. In the embodiments shown in the figures, the (side) walls such as those indicated in 1a and 1b meet the base and top of the microchannel (at 90° so that the microcavities have a rectangular or square cross-section, but in alternative embodiments, the micro structures may have a circular or oval cross-section for example, or a cross-section of other shape.

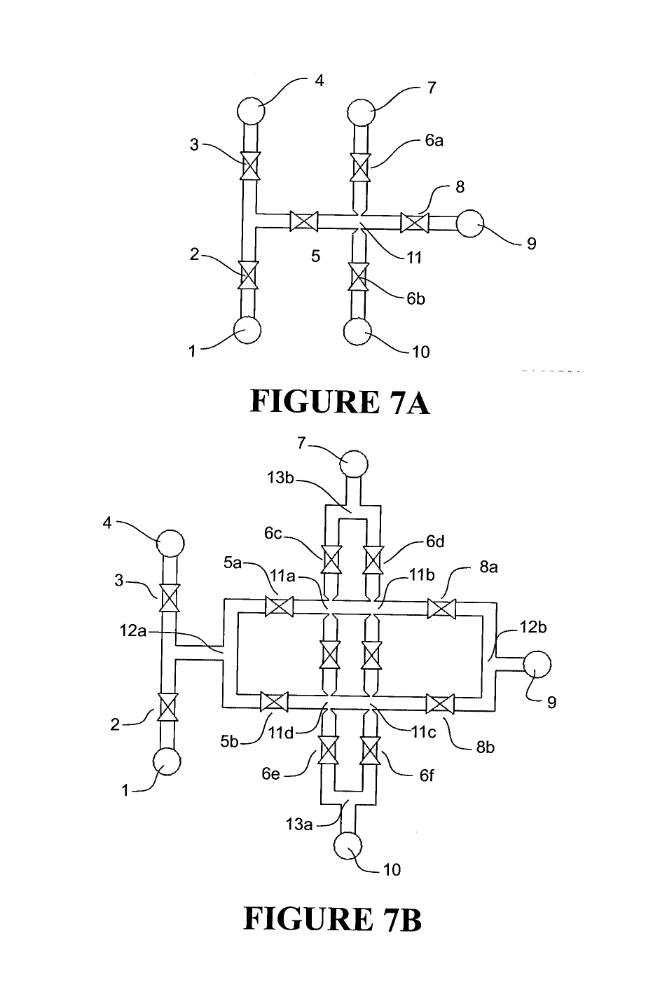

FIG. 7a schematic shows a microfluidic system on a chip that has two traps that operate according to the method described above. A summary of the inlets, outlets, and valves is given in Table 1. This system has inlets 1, 4, and 10 (I1, I2 and I3) and outlets 7 and 9 (O1 and O2). A sperm-supporting medium is connected to inlet 1 (I1) and inlet 8 (I3). The medium for I1 is used for flushing the injection channels and the medium for I3 is used for flushing the reservoirs and the two medium can be the same but do not necessarily need to be. The solution containing the sperm is connected to inlet 4 (I2). Outlet 7 (O1) enables separated sperm to be taken out of the microfluidic system. Outlet 9 (02) allows the waste flow to exit the microfluidic system. In addition, the pressure-driven flow of liquids in the microfluidic system between the inlets and outlets is controlled by coordinated switching of the valves to create the sequence of flow at a trap 11 as described above in FIGS. 3a to 3c. This is a preferentially a microfluidic device in which on-chip valves are used to control the flow within the channels. The valves may be a variation of “Quake” valves—in which a fluidic layer is actuated via an overlaying pneumatic/hydraulic control structure controlled off chip by computer controlled solenoid valves. Alternatively the valves may be akin to “normally off” “Folch” valves. Both are “multilayer microfluidic” and require the use of elastomeric materials such as polydimethylsiloxane (PDMS). Other valve systems can be implemented, for example by the use of off-chip actuators skin to those used for “braille” microfluidics—also based on PDMS. Other on-chip valve systems, including those not requiring elastomeric substrates may be used. Alternatively instead of valves fluid flow may be controlled by controlling a balance of pressures (e.g. pressure controlled pumps or hydrostatic pressure), by electrokinetic means (e.g. by electro-osmosis) or passive control (e.g. valves or gates created by careful fluidic geometry coupled with surface tension effects or differential surface energies). Alternatively again the valves may be non-integrated valves off-chip.

The system in FIG. 7a has valves 2, 3, 5, 6, and 8 (V1, V2, V3, V4, and V5. Four valve modes are used in the typical operation of the device. These modes are summarised in Table 2. In the inject mode valves 3, 5, and 8 (V2, V3, and V5) are open, i.e. they allow liquid flow, while the rest of the valves are closed, i.e. they do not allow liquid flow. In the sort mode all valves are closed. In the flush mode valves 2, 5, and 8 (V1, V3, and V5) are open and the rest of the valves are closed. In the collect mode V1 (2) and V4 (6) are open and the rest of the valves are closed.

In typical operation the microfluidic system will be connected to all inlet streams, outlet streams, and valve controls. The inlet pressures of inlets 1, 4 and 10 (I1, I2, and I3) are set to be greater than the outlet pressures at outlets 7 and 9 (O1 and 02). The fluid microchannels are then filled with medium by alternating between flush and collect modes to prime the fluidic system with the medium(s). The valve mode for inject is then set and the liquid containing the sperm is added to the injection channel(s). A zero-flow condition is then created by setting the sort mode and the sperm are allowed to sort into the trap structures. The time that the system is in the sort mode can be adjusted between 1 second and 30 minutes, or between 30 seconds and 300 seconds, or between 45 seconds and 120 seconds. The flush mode is then set to displace the solution containing unsorted components in the injection channel(s) with medium. The collect mode is then used to flow the sorted components through the collection outlet 7 (O1). Each cycle of inject, sort, flush, and collect sequences allows motility-sorted sperm to be collected from the system. Multiple cycles can be used to increase the total collection volume and hence the number of sperm sorted.

In addition a sub-cycle of inject, sort, and flush modes can be repeated in sequence for two or more times before setting the collect mode to enable concentration of sperm within the collection reservoirs. Also, during the flush mode the unsorted solution can be led to waste or used again. Also, in the two-trap design each collection channel can be collected individually by using the injection medium to flush the collection reservoir during the collect mode, i.e. collect from O1 by opening only valves 2, 5 (V1, V3), and a single collection valve 6a or 6b (V4) while keeping all other valves closed.

An alternative one-trap design can be implemented by eliminating one of the collection valves 6b (V4) and inlet 10 (I3) and instead using the injection medium to flush the collection reservoir during the collect mode, i.e. open valves 2 and 5 (V1 and V3), and the remaining collection valve 6a (V4).

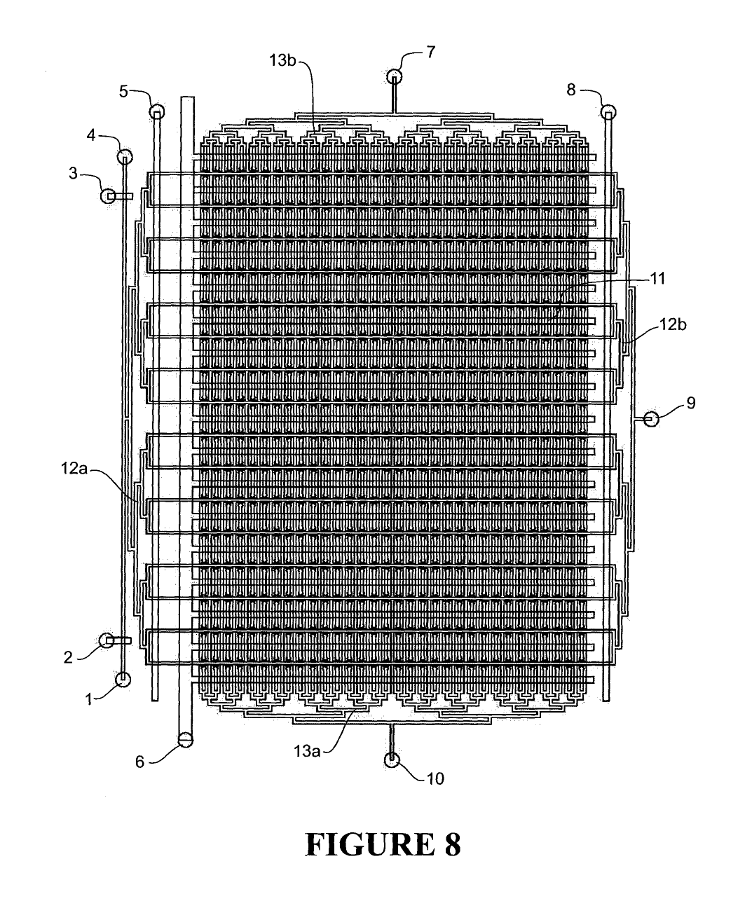

TABLE 1
Reference numbers, names, and descriptions
for FIGS. 7a, 7b, and 8

| # | Name | Description |
|---|---|---|
| 1 | I1 | Inlet 1, for flush medium |
| 2 | V1 | Inlet 1 Valve |
| 3 | V2 | Inlet 2 Valve |
| 4 | I2 | Inlet 2, for sperm solution |
| 5 | V3 | Inject Valve(s) |
| 6 | V4 | Trap Valve(s) |
| 7 | O1 | Outlet 1, for sample collection |
| 8 | V5 | Outlet 2 Valve(s) |
| 9 | O2 | Outlet 2, for waste |
| 10 | I3 | Inlet 3, for flush medium |
| 11 | Trap(s) | A microchannel group of at least one channel, trap and |
| | | reservoir |
| 12 | Multiplexed | Structures to join a single inlet or outlet to multiple |
| | Injection | injection channels |
| 13 | Multiplexed | Structures to join a single inlet or outlet to multiple |
| | Collection | collection channels |

TABLE 2
Microfluidic system operation modes and associated valve states

| | Valve State (X = closed, ◯ = open) | | | | |
|---|---|---|---|---|---|
| Mode | V1 | V2 | V3 | V4 | V5 |
| Inject | X | ◯ | ◯ | X | ◯ |
| Sort | X | X | X | X | X |
| Flush | ◯ | X | ◯ | X | ◯ |
| Collect | ◯ | X | X | ◯ | X |

FIG. 7b schematically shows a microfluidic system on a chip that has more than 2 traps that operate according to the method described above; this case shows a schematic design for 4 traps. The inlets, outlets, and operation are similar to the system in FIG. 7a, however the system in FIG. 7b has the injection channel multiplexed at the inlet 12a and outlet 12b such that multiple injection microchannels are created. The collection inlet 13a and outlet 13b are similarly multiplexed to create multiple collection microchannels and reservoirs. As a result of the multiple injection and collection channels there are multiple traps created 11a-11d. In order to create zero-flow conditions valves 5a and b and 6c-f (V3 and V4) are elaborated to close multiple injection and collection channels as indicated.

In addition, by multiplexing the injection and collection channel inlets and outlets microfluidic systems with an array of traps can be created with a large number of traps; preferably a total number of traps between 1 and 1,048,576, or between 16 and 32,768, or between 512 and 4096.

In addition, the manifolds for the injection channels 12 and collection channels 13 should preferentially provide substantially equal pressures across all entry points where the fluid leaves the manifold and enters the collection channels.

FIG. 8 schematically shows a microfluidic system on a chip that has 1024 traps (11) that operate according to the method described above in FIGS. 7a and 7b; the same number references are used.). In the schematic a valve is represented by a crossing of the channel indicated as a valve and the channels leading from the inlets and outlets. These valve channels and fluid channels do not articulate.

The invention is further illustrated by the following examples of laboratory work:

### EXAMPLES

### Example 1

### Thawed Bovine Sperm Sorting Efficiency

Methods:

Commercially available frozen bovine semen was sourced for testing from Livestock Improvement Corporation, New Zealand. A sperm sorting system of the invention generally as described with reference to FIGS. 7 and 8 was used, with 1024 sperm traps, a manifold-style collection system, pressure-driven flow, and on-chip valves to switch laminar fields and to create zero-flow conditions. The device was primed with HSOF medium at 37° C. prior to use. A straw of frozen semen, containing approximately 20 million sperm, was defrosted using a standard operation protocol, and sperm separated from the semen, resuspended in warmed HSOF medium and maintained in a foil-covered plastic tube at 37° C. An aliquot of washed sperm was then counted using a haematocytometer and its motility visually assessed. The washed sperm was then diluted using warmed HSOF medium to the required concentration (4 million/ml). The diluted sample was then loaded in to the microfluidic system and this fully automated set up sorted and collected highly motile sperm from the loaded sample at 10 second intervals. The concentration and motility of these samples were assessed.

Results:

Frozen-thawed semen samples from the same bull were sorted on four different occasions. Data from each run were based on the mean of 10 separate replicates. A summary of the concentration and motility of microfluidically sorted sperm from all four runs is shown below in Table 3.

TABLE 3
Summary of the concentration and motility of sperm
sorted by the microfluidic device in comparison with
those of the original pre-sorted sperm and in relation
to the industry target required for IVF.

| | Thawed pre- | Microfluidic | Target for |
|---|---|---|---|
| Measure | sorted sperm | sorted sperm | IVF |
| Concentration of | 20 | 1.7 | 1.0 |
| motile sperm | | | |
| (×10⁶/ml) | | | |
| Percentage of | 50% | 90+% | 90% |
| motile sperm | | | |
| Motility Grade* | Mixed | Progressive (a) | Progressive (a-b) |
| | (a-d) | | |
| Throughput | N/A | 1,720,000 | 300,000 |
| (sperm/30 min) | | | |

*Motility grades based on World Health Organisation (WHO) Criteria 2010; a = fast progressive, b = slow progressive, c = non-progressive, d = immotile.

Conclusions:

The microfluidic system was able to successfully separate highly motile sperm when compared to the pre-sorted sample. In addition, the concentration and motility of the separated sperm were comparable, and even greater in terms of concentration, to the industry target used in IVF.

### Example 2

### Fresh Human Sperm Sorting Efficiency

Methods:

Sperm samples were donated from male patients (n=17) receiving fertility treatment at Repromed (Adelaide, South Australia). Samples were collected using standard clinical methods (Bakos et al., 2011, Fertility and Sterility, 95, 1700-1704), allowed to liquefy for 30 minutes at room temperature, before an aliquot was subjected to initial visual assessment for motility and then counted using a haematocytometer. Neat samples were divided into three. These were processed following routinely undertaken density-gradient (Bakos et al., 2011) and swim-up (Zhang et al., 2012, Human Fertility, 14, 187-191) methods for human sperm or by the microfluidic system detailed in Example 1. Neat samples were loaded on to the microfluidic system, whilst samples were diluted in commercially available medium for processing via the two standard methods. The concentration, motility and morphology of these samples were assessed and compared between the three different methods.

Results:

The characteristics of a representative fresh semen and microfluidic sorted sample compared with the target characteristics required for human IVF sperm are shown in Table 4. For all measures, microfluidic sorted sperm are comparable or far exceed (throughput of sperm) targets for human IVF.

TABLE 4
The concentration and motility of sperm sorted by the microfluidic
device in comparison with that of the original fresh sample of
a patient indicative of those included in the study. Industry
targets for human IVF are also included for reference.

| | | Microfluidic | Target for |
|---|---|---|---|
| Measure | Fresh Semen | sorted sperm | Human IVF |
| Concentration of | 20.0 | 2.3 | 1.0 |
| motile sperm | | | |
| (×10⁶/ml) | | | |
| Percentage of | 57% | 91% | 90% |
| motile sperm | | | |
| Motility Grade* | Mixed | Progressive (a) | Progressive (a-b) |
| | (a-d) | | |
| Throughput | N/A | 1,920,000 | 300,000 |
| (sperm/30 min) | | | |

*Motility grades based on World Health Organisation (WHO) Criteria 2010; a = fast progressive, b = slow progressive, c = non-progressive, d = immotile.

Sperm throughput (sperm/second) was over 5-fold greater and consequently, the time taken to sort 300,000 sperm was 13-fold quicker for microfluidically sorted sperm than those sorted either by density gradient or swim-up (Table 5). The output of the microfluidic device was 449 nl/sec of motility sorted sperm in exchanged medium. Concentrations of 3.25M/ml, 3.0M/ml and 2.7M/ml of sperm were recovered after processing by the microfluidic system, density gradient and swim-up, respectively.

TABLE 5
Summary of the throughput (total number of motile sperm processed
in a determined time), time taken to reach the IVF target (300,000
motile sperm) and the percentage of normal morphological grade
of sperm processed from fresh samples (n = 6) by the three
methods of density-gradient, swim-up and microfluidic chip.

| | | | Micofluidic |
|---|---|---|---|
| Measure | Density-gradient | Swim-up | sorted sperm |
| Throughput of | 242 | 250 | 1309 |
| motile sperm | | | |
| (sperm/second) | | | |
| Time to reach target of | 62 | 54 | 4 |
| 300,000 motile sperm | | | |
| (mins) | | | |

Samples processed were from patients with various causes of male factor infertility, as well as those deemed to have ‘normal’ sperm samples, according to the WHO criteria (2010). For all three methods tested, the mean concentration of sperm recovered exceeded the target for human IVF (1 million/ml), as summarised in Table 6. In seven of the 17 samples higher concentration of sperm were recovered in the microfluidic sorted sperm compared with the swim-up method, however, overall the mean concentration was higher using the swim-up versus the microfluidic method. Comparable motility percentages were evident for the density gradient and swim-up methods, with slightly lower percentages in the microfluidic samples. All were around the 90% target used in IVF clinics. Normal morphology percentages were comparable between all three methods, although slightly higher for those samples processed by density gradient (Table 6).

TABLE 6
Summary of the mean concentration (million/ml), motility
(%) and morphological grade (% normal) of sperm processed
from fresh human samples (n = 17) by the three methods
of density-gradient, swim-up and microfluidic chip.

| | | | Microfluidic |
|---|---|---|---|
| Measure | Density-gradient | Swim-up | sorted sperm |
| Mean concentration | 18.7 | 6.9 | 3.8 |
| (million/ml) | | | |
| Mean percentage | 93.4 | 93.3 | 86.5* |
| motility (%)¹ | | | |
| Mean | 12.8 | 11.0 | 11.4 |
| morphology grade | | | |
| (% normal)² | | | |

¹Motility (fast and slow progressive) and
²morphology assessment based on World Health Organisation (WHO) Criteria 2010.
*Mean of n = 15 samples.

Conclusions:

The microfluidic system was able to successfully separate highly motile human sperm when compared to the pre-sorted sample. The throughput and speed of processing of highly motile human sperm by the microfluidic system was far greater when compared to the standard methods of density gradient and swim-up currently used. Density gradient and swim-up methods produced more concentrated samples on average, with slightly higher motility but comparable normal morphology to microfluidic sorted samples. However, the concentration and motility of fresh human sperm separated by all three methods were much greater than the industry target used in human IVF.

### Example 3

### Bovine Oocyte Fertilisation Ability

Methods:

Microfluidically sorted sperm were prepared as described in example one, whilst control unsorted sperm were prepared following a standard IVF Percoll wash protocol (Kimura et al., 2004, Molecular Reproduction and Development, 68, 88-95). Oocytes were obtained for abattoir recovered bovine ovaries, processed and cultured prior to fertilization as outlined in Kimura et al. (2004). The concentration and motility of sperm was determined prior to addition to the oocytes. Sperm were diluted to result in a final concentration of 1 million/ml per media drop. The number of oocytes per medium drop was standardized (5 per drop) and equivalent between the control and microfluidically sorted sperm groups. In accordance with methods and medium used in Kimura et al. (2004), the day 3 cleavage rate (number of embryos on day 3/number of oocytes in culture; Day 0=fertilisation) and day 7 blastocyst rate (number of blastocysts on day 7/number of oocytes) were recorded. Three separate culture replicates were undertaken.

Results:

Despite day 3 cleavage rates being similar between the two methods, a significantly greater number of oocytes developed to embryos in the group fertilised with microfluidically sorted rather than control sperm. Day 7 embryos fertilised using either control or microfluidic sorted sperm were of comparable quality (Table 7).

TABLE 7
Summary of data from the in vitro fertilisation
studies comprising of three separate cultures.

| | | Microfluidic | Significance |
|---|---|---|---|
| Measure | Control | sorted sperm | level |
| Sperm Concentration | 1.0 | 1.0 | NS |
| for IVP (×10⁶/mL) | | | |
| Number of oocytes | 559 | 583 | NS |
| Day 3 Cleavage rate | 88% | 89% | NS |
| Number of | 121 | 192 | P < 0.01 |
| Day 7 Blastocysts | | | |
| Day 7 Blastocyst rate | 22% | 33% | P < 0.01 |
| Percentage of good quality* | 67% | 68% | NS |
| Day 7 embryos | | | |

*Blastocyst grades 1 and 2 only, based on recognised assessment criteria by the International Embryo Transfer Society (IETS).
NS = not-significant (P > 0.05).

Conclusions:

These data indicate that the microfluidically sorted sperm generate equivalent cleavage rates but an increased number of comparable quality day 7 embryos compared with sperm processed using current standard methods.

1. (canceled)

2. A method of separating motile and non-motile sperm which comprises:
delivering a fluid containing the sperm in a fluid into a microchannel comprising a wall which includes a wall termination or a change in angle away from the microchannel (wall termination),
allowing motile sperm to move in substantially no-flow conditions in the microchannel along the wall and to exit the microchannel by changing direction away from the microchannel at or near the wall termination, to or towards a collection reservoir, and
recovering fluid containing the motile sperm from the collection reservoir.

3. A method according to claim 2 wherein the change in angle is between about 20° and approaching 180°.

4. A method according to claim 2 wherein the microvolume is further defined by at least one further wall termination adjacent the first said wall termination, the two or more wall terminations defining between them an opening from the microvolume.

5. A method according to claim 4 wherein the opening is to a collection reservoir for motile sperm.

6. A method according to claim 2 wherein allowing motile sperm to move in substantially no flow conditions or allowing at least some motile sperm to move along said wall and to exit the microvolume includes maintaining the substantially no flow conditions for at least 10 seconds.

7. (canceled)

8. A method according to claim 2 carried out on a microfluidic chip and wherein said microvolume is a microchannel on said microfluidic chip.

9. A system for separating motile and non-motile sperm, which comprises a microvolume into which a fluid containing sperm can be delivered, at least partly defined by a wall which includes a termination or a change in angle away from the microvolume (‘wall termination’), the wall termination at least in part defining an exit from the microvolume to or towards a collection reservoir or passage from the microvolume for motile sperm.

10. (canceled)

11. A system according to claim 9 wherein the change in angle is between about 20° and approaching 180°.

12.-16. (canceled)

17. A system according to claim 9 wherein the microvolume is further defined by at least one further wall termination adjacent the first said wall termination, the two or more wall terminations defining between them an opening from the microvolume.

18. A system according to claim 17 wherein the opening has a dimension in a length of the microchannel of at least about 10 microns.

19. A system according to claim 17 wherein the opening has a dimension in a length of the microchannel of up to about 750 microns.

20. A system according to claim 17 wherein the opening is to a collection reservoir for motile sperm.

21. A system according to claim 17 wherein the opening is to a collection reservoir for motile sperm defined in least in part by a concavely arcuate wall portion to assist in retaining motile sperm within the collection reservoir.

22.-23. (canceled)

24. A system according to claim 9 wherein a length of the microvolume comprises an extended channel structure.

25. A system according to claim 9 wherein the microvolume has a width in the range about 10 to about 5000 microns.

26. (canceled)

27. A system according to claim 9 wherein the microvolume has a depth in the range about 5 to about 1000 microns.

28.-30. (canceled)

31. A system according to claim 9 on a microfluidic chip and wherein said microvolume is a microchannel on said microfluidic chip.

32. A system according to claim 9 including a control system arranged to control:
delivery of fluid containing sperm in a fluid into the microvolume,
subsequent maintenance of a period of substantially no-flow conditions in the microvolume, and
recovery or delivery of fluid containing the motile sperm from the microvolume after a period of said substantially no-flow conditions.

33. A system according to claim 32 wherein said period of substantially no flow conditions is at least 10 seconds.

34. (canceled)

35. A microfluidic system for separating motile and non-motile sperm, which comprises:
a microchannel into which a fluid containing sperm can be delivered,
a collection reservoir for motile sperm defined in least in part by a concavely arcuate wall portion to assist in retaining motile sperm within the collection reservoir.
a first termination or a change in angle away in a wall in part defining the microchannel (‘first wall termination’),
a second termination or a change in angle away in said wall further along said wall in a flow direction (‘second wall termination’),
an exit opening from the microchannel to the collection reservoir defined between said first wall termination and said first wall termination,
said microfluidic system including a control system arranged to control:
delivery of fluid containing sperm in a fluid into the microchannel,
subsequent maintenance of a period of substantially no-flow conditions in the microchannel, and
subsequent flushing with fluid of the microchannel after the period of said substantially no-flow conditions.

36. (canceled)

37. A multichannel microfluidic array for separating or concentrating motile sperm, said array comprising:
an inlet port for sperm;
an inlet port for carrier medium;
an exit port for carrier medium and unseparated or non-motile sperm;
a delivery port for flushing medium;
a recovery port for separated or concentrated motile sperm;
a plurality of parallel microchannels, each microchannel including side walls, and each side wall having a plurality of wall terminations; each wall termination forming an entrance to a motile sperm collection reservoir;
a plurality of sperm collection reservoirs interconnectable between the delivery port and the recovery port through the microchannels;
valve means for controlling fluid flow either from the inlet ports to the exit port or from the delivery port to the recovery port; and
manifold systems to distribute and recover fluids substantially evenly to and from the microchannels and/or collection reservoirs.
